(19) United States
(12) Patent Application Publication
(10) Pub. No.: US 2001/0016704 A1
(43) Pub. Date: Aug. 23, 2001

(54) LOW PROFILE FLUID DELIVERY AND SEALING SYSTEM FOR A CATHETER

(76) Inventors: GHOLAM REZA ZADNO-AZIZI, NEWARK, CA (US); ISAAC J. KIM, SAN JOSE, CA (US); CELSO J. BAGAOISAN, UNION CITY, CA (US); GEORGE TSAI, SUNNYVALE, CA (US); SAMUEL L. OMALEKI, MORGAN HILL, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/025,990

(22) Filed: Feb. 19, 1998

(51) Int. Cl. A61M 29/00

(52) U.S. Cl. 604/99.02; 604/256

(57) ABSTRACT

A plug system for a low profile hollow catheter or other guidewire or balloon catheter, which catheter has a distal portion and a proximal portion and at least one lumen in fluid communication therebetween. The catheter further has an outer diameter in the range of about 0.01″ to 0.04″ and an inner diameter in the range of about 0.007″ to 0.035″. The plug system comprises an elastomeric plug for engaging and sealing a proximal open end of the catheter in such a way that the maximum outer diameter of the plug does not exceed the outer diameter of the catheter. A low profile fluid delivery and sealing system for a catheter with a lumen comprises a pierceable and self-resealable member sealably coupled to the lumen at a proximal portion of the catheter forming an assembly. The transverse cross sectional area of the assembly as viewed along a longitudinal axis of the catheter does not exceed 150% of the original transverse cross sectional area of the catheter.

### BACKGROUND OF THE INVENTION

1. Field of the Invention

The present invention relates to a low profile fluid delivery and sealing system for delivering a fluid to a low profile catheter, and, more particularly, for inflating or deflating a balloon on a guide or other catheter.

2. Description of the Related Art

Guidewires or guide catheters are conventionally used to guide the insertion of various medical instruments, such as catheters, to a desired treatment location within a patient's vasculature. In a typical procedure, the clinician forms an access point for the guidewire by creating an opening in a peripheral blood vessel, such as the femoral artery. The highly flexible guidewire is then introduced through the opening into the peripheral blood vessel, and is then advanced by the clinician through the patient's blood vessels until the guidewire extends across the vessel segment to be treated. Various treatment catheters, such as a balloon dilatation catheter for a percutaneous transluminal coronary angioplasty, may then be inserted over the guidewire and similarly advanced through vasculature until they reach the treatment site.

In certain treatment procedures, it is desirable to successively introduce and then remove a number of different treatment catheters over a guidewire that has been placed in a particular location. In other words, one treatment catheter is “exchanged” for another over a single guidewire. Such an exchange typically involves withdrawing the treatment catheter over the guidewire until the treatment catheter is fully removed from the patient and the portion of the guidewire which extends from the patient. The guidewire is then available to act as a guide for a different treatment catheter.

In an emboli containment device, which may utilize an occlusion balloon, it may be desirable to exchange therapeutic catheters without deflating the occlusion balloon. Further, in another type of catheter it is sometimes advantageous to anchor the guidewire during the exchange using a distal inflatable balloon. As can be readily appreciated, the withdrawal of treatment catheters over a placed guidewire may result in the guidewire being displaced from its position. To overcome this difficulty, the prior art has developed “anchorable” guidewires, which generally feature some structure on their distal ends to releasably secure the guidewire at a particular location in the patient for the duration of the medical procedure. One such anchorable guidewire is disclosed in U.S. Pat. No. 5,167,239 to Cohen et al., which discloses a hollow guidewire with an inflation lumen and an expandable balloon on its end. The Cohen guidewire is positioned in the same manner as a conventional wire guidewire, but once placed, its expandable balloon is inflated to contact the surrounding vasculature, thereby preventing the guidewire from being displaced.

Because a permanent inflation manifold, of the type used with conventional catheters having an inflatable balloon, would prevent other catheters from being inserted over the Cohen guidewire, the Cohen device also includes a removable inflation manifold, and a check valve to maintain the balloon in the inflated state when the manifold is removed. The check valve apparatus used by the Cohen device is relatively bulky, and is described as having an outer diameter in its preferred embodiment of 0.0355 inches. Consequently, any treatment catheter intended to be inserted over the Cohen device must have an interior guidewire lumen larger than the outer diameter of the Cohen valve, which for the preferred embodiment, requires an interior lumen with a diameter of more than 0.0355 inches.

As is readily appreciated by those of skill in the art, increasing the interior lumen size of a treatment catheter results in an increase in the outer diameter of the treatment catheter. For treatment procedures which take place in vasculature having a large blood vessel diameter, such as iliac arteries, a treatment catheter guidewire lumen of a size necessary to accommodate devices such as those described by Cohen would have little or no affect on the ability of the catheter to fit within the blood vessel. However, many blood vessels, where it is desirable to apply a catheter treatment, are quite narrow. For example, the left coronary arteries are blood vessels having diameters ranging from 2 to 4 mm, and are susceptible to plaque. It would be desirable to use a catheter exchange treatment procedure, such as angioplasty, to treat such lesions, but the narrow diameter of the coronary vessels makes use of anchorable guidewires having large valve diameters impractical.

In other applications, it may be desirable to deliver fluids through a low profile, hollow guidewire or similar low profile catheter. Such fluids may provide irrigation, blood perfusion, drug delivery, and the like, to the treatment site.

Consequently, there exists a need for a very low profile fluid delivery and sealing system which can be used with a hollow guidewire or a catheter. The present invention provides such a system.

### SUMMARY OF THE INVENTION

According to one aspect of the present invention, a plug system for a guide catheter or a balloon catheter is provided. The catheter has a balloon thereon and at least one lumen in fluid communication with the balloon. Preferably, the catheter has an outer diameter in the range of about 0.01″ to 0.041″, and an inner diameter in the range of about 0.007″ to 0.035″. The plug system comprises an elastomeric plug for engaging and sealing a proximal open end or other opening in the proximal portion of the catheter in such a way that the maximum outer diameter of the plug does not exceed 140% of the outer diameter of the catheter.

According to another aspect of the present invention, a low profile fluid delivery and sealing system for a catheter with a lumen is provided. This low profile system comprises a pierceable and self-resealable member sealably coupled to the lumen at a proximal open end or other opening in the proximal portion of the catheter forming an assembly. The transverse cross sectional area of the assembly as viewed along a longitudinal axis of the catheter does not exceed substantially the original transverse cross sectional area of the catheter, and preferably is less than such cross-sectional area while allowing accessibility to the lumen through the member. In cases where the cross-sectional area of the assembly is greater than that of the original cross-sectional area, it should be minimized so as to be, for example, less than 150% thereof.

According to still another aspect of the present invention, a method for sealing or delivering a fluid into a lumen of a catheter is provided. The catheter has an opening in fluid communication with the lumen. The method comprises the steps of: a) providing an elastomeric member to seal the opening of the lumen of the catheter; b) providing a syringe with a needle, and inserting the needle into the lumen through the elastomeric member and the opening so as to establish fluid communication between the lumen and the syringe; and c) removing the needle to allow the elastomeric member to self-seal. Another step of guiding the needle into the sealed lumen is disclosed.

In another aspect of the present invention, there are provided specially designed adaptors and syringes which can be used with the fluid delivering and sealing system of the present invention.

This invention allows a balloon to be inflated and kept inflated with minimum or no increase in catheter profile. Alternatively, the catheter can be used for other fluid delivery applications. The system of the present invention is simple and reliable with minimal mechanism and parts, which greatly reduces the manufacturing cost and the risk of error during a surgical operation.

This invention is preferably used with guidewires and catheters with low profile. However, it can be also used with other tubings of small diameters.

### BRIEF DESCRIPTION OF THE DRAWINGS

FIG. 1 shows a cross-sectional view of a plug system according to one embodiment of the present invention;

FIG. 2 shows an exploded view of the plug system of FIG. 1;

FIG. 3A shows a cross-sectional view along the longitudinal axis a catheter with a plug in accordance with another embodiment of the present invention;

FIGS. 3B, 3B-1 and 3C show a cross-sectional view of a plugged catheter with two lumens in accordance with other embodiments of the present invention.

FIG. 4A shows a cross-sectional view of a catheter coated with a plugging layer in accordance with one embodiment of the present invention.

FIG. 4B shows a cross-sectional view of a catheter with two lumens coated with a plugging layer in accordance with one embodiment of the present invention.

FIG. 4C shows a cross-sectional view of the catheter of FIG. 4B as viewed along 4c-4c direction accordance with one embodiment of the present invention.

FIG. 5 shows a cross-sectional view of a catheter, a portion of which is replaced with a pierceable member in accordance with one embodiment of the present invention.

FIG. 6 shows an adaptor used for the catheter shown in FIG. 4B.

FIGS. 7A, 7B, 7C and 7D show a perfusion catheter with two lumens and an adaptor used for the catheter in accordance with one embodiment of the present invention.

FIG. 8 shows a cross-sectional view along the longitudinal axis of a low volume syringe according to one embodiment of the present invention.

FIG. 8A shows a cross-sectional schematic view of the stop mechanism of the low volume syringe of FIG. 8.

FIG. 9 shows an end view of the low volume syringe of FIG. 8;

FIG. 10 shows a syringe having an alternative embodiment of a stop member.

FIGS. 11A and 11B show a syringe having yet another embodiment of a stop member.

FIG. 12 shows a preferred embodiment of a high pressure syringe assembly with an exploded view of the plunger stop mechanism;

### DETAILED DESCRIPTION OF THE PREFERRED EMBODIMENTS

In a preferred aspect, the present invention provides a plugging and sealing mechanism for a low profile guide catheter having an occlusion balloon or other medical balloon devices or other catheters. The mechanism can keep the balloon inflated and keep the pressure of the balloon. In the meantime, the plugging mechanism does not increase or just slightly increase the overall profile of the catheter so that exchange of devices over the catheter can be conducted without removing the plugging mechanism.

However, it will be recognized that the principals of the present invention apply equally well to other fluid delivery applications, such as irrigation, blood perfusion, drug delivery and the like. Moreover, as described herein, “plug” or “seal,” and the like, may have reference to either the complete or partial blockage of fluid communication in the hollow catheter, as is appropriate for the particular application. As described herein, the plugging mechanism can be applied at various locations on the catheter, including the proximal open end, side openings or other ports, and the like.

According to one aspect of the present invention, the plugging mechanism can be a plug inserted into a lumen of a guidewire or a catheter which seals the lumen usually at the proximal end of the catheter. The lumen can be in fluid communication with a balloon located near the distal end of the catheter. The plug can be made of various forms. For example, it can be an elongated solid body with a cross section matching the cross section of the lumen. It may also have a cap-like shape with side walls defining a space for receiving the proximal end of the catheter. In the case of the elongated solid plug, the profile of the catheter remains the same after the plug is inserted into the lumen. When a cap like plug is used, the profile of the catheter at the conjunction area between the catheter and the cap-like plug is increased by 2 times thickness of the side wall of the cap-like plug. Therefore, the thickness of the side wall needs to be minimized in order to keep a low profile. But the side wall has to be thick enough to provide the strength for holding the plug in position and sealing the open end of the catheter. Preferably, the side wall thickness is less than 0.005″.

The plug usually seals the open end of the catheter. In order to deliver a fluid to the lumen, for example, to inflate a balloon, a needle is pierced through the plug into the lumen to inject an inflation fluid. In view of the small cross sectional area, the length or thickness of the plug in the direction of the needle piercing should be large enough to provide enough material, so that when the needle is removed from the plug, the piercing pass in the plug can be resealed due to the elasticity of the plug material. The thickness usually is in the range of about 0.05 to 0.5″.

The plugging mechanism of the present invention can be also provided as a sealing or plugging layer or membrane. In this case, the side wall of a catheter has an opening. A sealing layer is secured around the opening so that the opening is covered and sealed by the sealing layer. Again the thickness of the sealing layer is preferably less than 0.005″. It can be also made of a tube shape having a diameter similar to that of the catheter. The tube is connected into the catheter at its two ends and becomes a portion of the catheter.

Any suitable biocompatible materials which reseal itself after a piercing needle is removed, and which stand a pressure of up to 20 atm or higher resulting from the inflation fluid inside the catheter, can be used for the plug. Examples of suitable materials include silicon, Urethane, latex, and C-flex. The plug may be molded or extruded, and attached to the catheter by adhesives such as cyanoacrylate. Other materials such as gels (silicone gels or other type) can also be used. As an example, the plug can be injection molded with C-flex elastomer (20A to 70A Shone A Hardness). C-flex elastomer and silicone can be extruded as solid rod and cut to appropriate length. Silicone plug can be also molded into plug shape by liquid injection molding. In this case, two liquid components of silicone are mixed just prior to injection into a hot mold. Latex and Urethane can be molded by dip-coating method. A core of appropriate shape is dipped into a hot liquid resin, removed and cooled. Then the plug of a shape corresponding to that of the core is stripped off the core.

Also, the plug system of the present invention can be used for catheters of various sizes. Catheters with an outer diameter in the range of about 0.01-0.04″ and an inner diameter in the range of about 0.007-0.035″ are preferred. More preferably, the catheter has an outer diameter in the range of about 0.01-0.021″ and an inner diameter in the range of about 0.007-0.017″. The catheter can be made of stainless steel, polyethylene, Pebax, Nitinol, or any other suitable materials.

Due to its small dimension, it is potentially difficult to insert a needle through the plug to an accurate position. Thus, in another aspect of the present invention, adaptors contain a guiding mechanism to guide the needle to the piercing point. It is also important to deliver an accurate amount of fluid to a catheter, particularly, when a low profile catheter is involved. Syringes capable of accurately delivering a small volume of fluid are provided. Those syringes can be used in combination with the adaptors.

The plug and sealing mechanism of the present invention will be described in more detail by referring to the drawings. Also provided in the present invention are adaptors and syringes which can be used with the plugging and sealing mechanism.

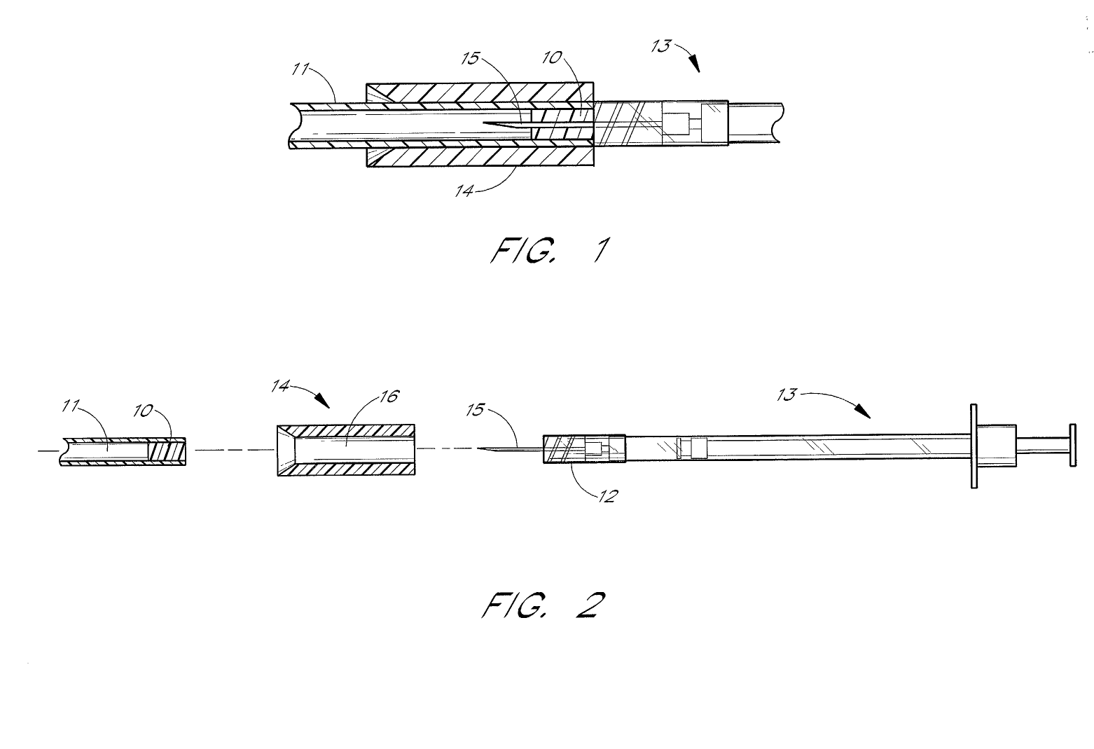

As shown in FIGS. 1 and 2, in one embodiment of the present invention, the plug system includes a plug 10 which is inserted into an open end of a lumen of a catheter 11 when in use. The lumen is in fluid communication with the balloon on the catheter 11. Plug 10 should be pierceable by a needle. Preferably, plug 10 is made of elastomeric material such as latex, silicone or C-Flex. The plug can be of any proper shape and dimension as long as they match with those of the catheter. As in most cases, the lumen of catheter 11 has a cylindrical shape, as does the plug. The outer diameter of plug 10 should match with the inner diameter of the catheter 11, that is, the outer diameter of plug 10 should be large enough so that when it is inserted into catheter 11 a desired degree of seal, such as a fluid-tight seal between the plug 10 and the inner surface of the lumen of catheter 11 can be achieved. However, the plug 10 should be able to be inserted into the lumen of the catheter 11 conveniently. In order to facilitate the insertion of plug 10, plug 10 can be tapered at the inserting end, or plug 10 can be made of a truncated conical shape. The entire plug 10 does not have to be inserted into the catheter 11. The length of plug 10 being inserted into the catheter 11 is determined by the sealing requirement and the pierceability of the needle. The maximal length of the plug is limited by the length of the needle and the minimal length is limited by the sealing requirement. For example, the plug may have a length in the range of about 0.05 to 0.5″. Alternatively, plug 10 can be built into the lumen of catheter 11, for example, by molding.

Also shown in FIGS. 1 and 2 is a syringe 13 having a needle 15. Needle 15 can be any proper conventional needle. Syringe 13 can be any proper type of syringes and can be manually or automatically controlled. At one end of the syringe body where needle 15 is attached to, it is provided with a male thread 12 for engaging an injection guide 14. Syringe 13 and injection guide 14 can also be attached to each other by other means, such as glue. Injection guide 14 has an elongated body with an aperture 16 along the longitudinal axis of the injection guide. Injection guide 14 is designed to help align plug 10 inserted in catheter 11 with needle 15. The shape of aperture 16 of injection guide 14 is designed to match the outer shape at one end of the catheter 11 so that the catheter 11 can be inserted into aperture 16 easily and move smoothly therein along the longitudinal axis of aperture 16. In the case of a cylindrical catheter, the aperture should be cylindrical and have an inner diameter slightly larger than the outer diameter of catheter 11. Preferably, aperture 16 is flared at one end where the catheter 11 is received. Injection guide 14 can be made of rigid material such as plastics.

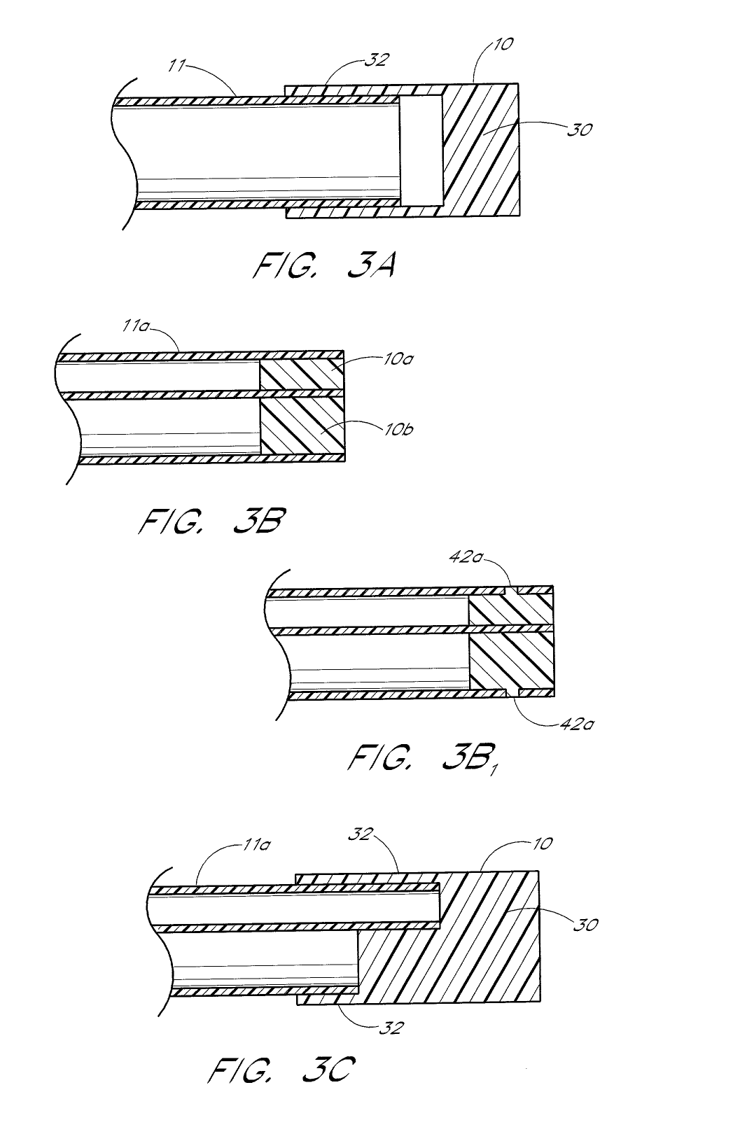

FIGS. 3A-3C show several different examples of the plug system. FIG. 3A shows a cap plug 10 having a body 30 and a side wall 32. Side wall 32 defines a space for receiving an open end of the catheter 11. The thickness of the body 30 is preferably in the range of about 0.05 to 0.5″ depending on the diameter of the catheter 11. Side wall 32 is secured and sealed to the open end, for example, by glue or fusion. The thickness of the side wall 32 is preferably less than 0.005″. The outer diameter the plug 10 should be minimized, and should not exceed 150% of the original outer diameter of the catheter 11.

FIG. 3B shows a catheter 11a which has two lumens. Each lumen has a plug (10a and 10b). Plugs 10a and 10b are similar to those plugs described previously. It is also possible to provide a hole or several holes 42a on the walls of the catheter 11 or 11a at the portion which engages plugs 10a and 10b, as shown in FIG. 3B-1. Hole 42a creates a lock for the plug and, thus, helps to secure the plug into the catheter. FIG. 3C shows a catheter 11a having two lumens. A cap plug is provided to plug and seal the open ends of the two lumens. Similarly, the catheter-plug assemblies shown in FIGS. 3A and 3C may be provided with hole 42a on the wall of the catheter to better secure the plug.

FIGS. 4A and 4B show two other examples of the plug mechanism of the present invention. In these embodiments, an opening is provided on the shaft wall of the catheter and covered with a plugging or sealing layer. FIG. 4A shows a catheter 11 with one lumen. There is an opening 42 provided on the shaft wall of catheter 11. Opening 42 is covered with a sealing layer 40. Layer 40 can be made of the same material as plug 10, and preferably is glued or fused to the shaft wall of the catheter 11. The thickness of the sealing layer 40 is preferably less than 0.005″ or less than 40% of the outer diameter of the catheter 11. FIG. 4B shows a catheter 11a with two lumens. Each lumen is connected to an opening 42, respectively. Openings 42 are covered and sealed by a sealing layer 40. In use a needle is pierced through the sealing layer 40 and the opening 42 into the lumen to inject a fluid such as an inflation fluid into the lumen and a balloon connected with the lumen. As will be discussed later, an adaptor is provided in the present invention to guide a syringe to the opening 42 because the opening has a small dimension and is not visible from the outside.

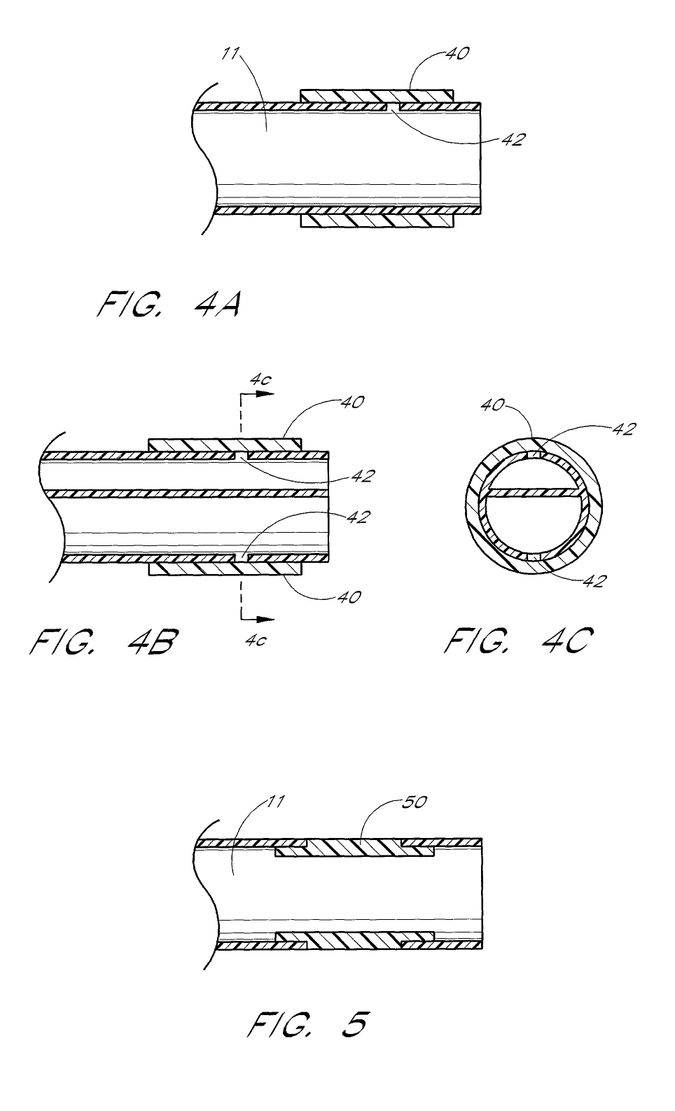

FIG. 5 shows another example of the plug and sealing mechanism of the present invention. In this embodiment, a tube of pierceable material having a similar diameter to that of a catheter 11 is inserted into the catheter 11 and forms a portion of the catheter. Each of the two open ends of the tube 50 is preferably glued or fused to an open end of the catheter 11. The materials suitable for plug 10 can be also used for the tube 50. But a greater strength is desirable for the tube 50.

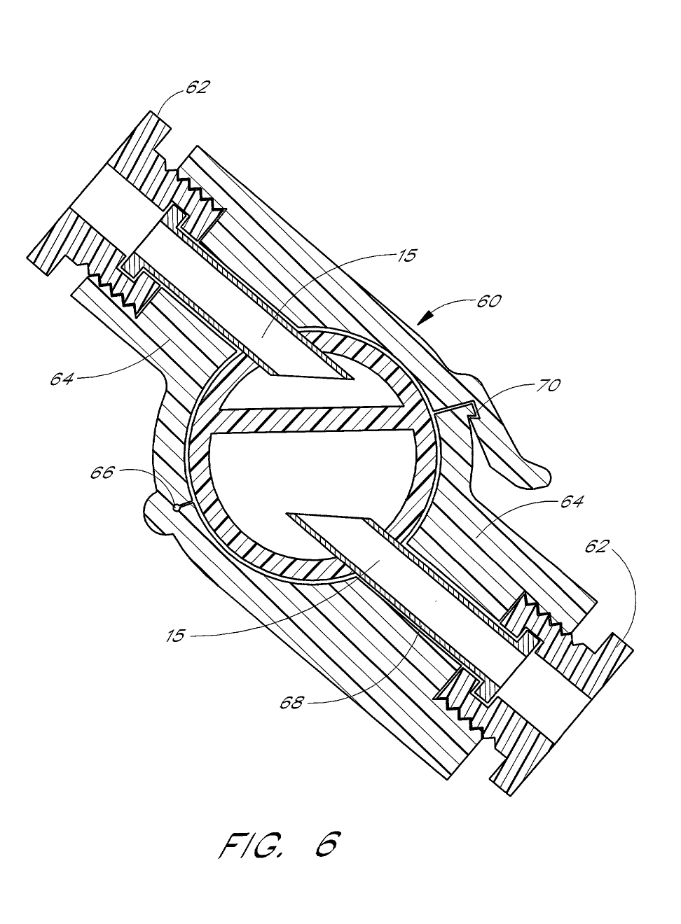

FIG. 6 shows an adaptor which can be used with a catheter having side openings or ports, similar to those shown in FIGS. 4 and 5. The adaptor 60 has two wrap-around, or side accessible, or open half shells 64. Thus, adaptor 60 is removable and replaceable. The two half shells 64 can be joined together through a hinged section 66. Each half shell 64 has a tapered and sharp needle 15 that rides inside a guide channel 68. The angle between the guide channel 68 and the surface of the shaft of the catheter 11a and the tip taper of the needle 15 are designed to achieve optimal penetration force and cut. The needle 15 can be connected to a threaded Luer 62. A push button or screw drive mechanism can be used to push the needle 15 inside the catheter 11a to a predetermined length. A locking mechanism 70 is provided to lock the adaptor 60 in position. As shown, the locking mechanism 70 can be formed by a projection extending along the open edge of a half shell 64 in the direction parallel to the axis of the catheter 11a, and a notch extending along the open edge of the other half shell 64 in the direction parallel to the axis of the catheter 11a. The notch engages the projection to hold the adaptor 60 on the catheter 11a when the two half shells 64 are brought together. The adaptor 60 can be made of metal such as stainless steel or rigid plastics such as polycarbonate. There are either visual markers on the catheter that needs to be aligned with in the adaptor or other stop mechanism that helps align the opening 42 of the catheter with the guide channel 68 of the adaptor 60.

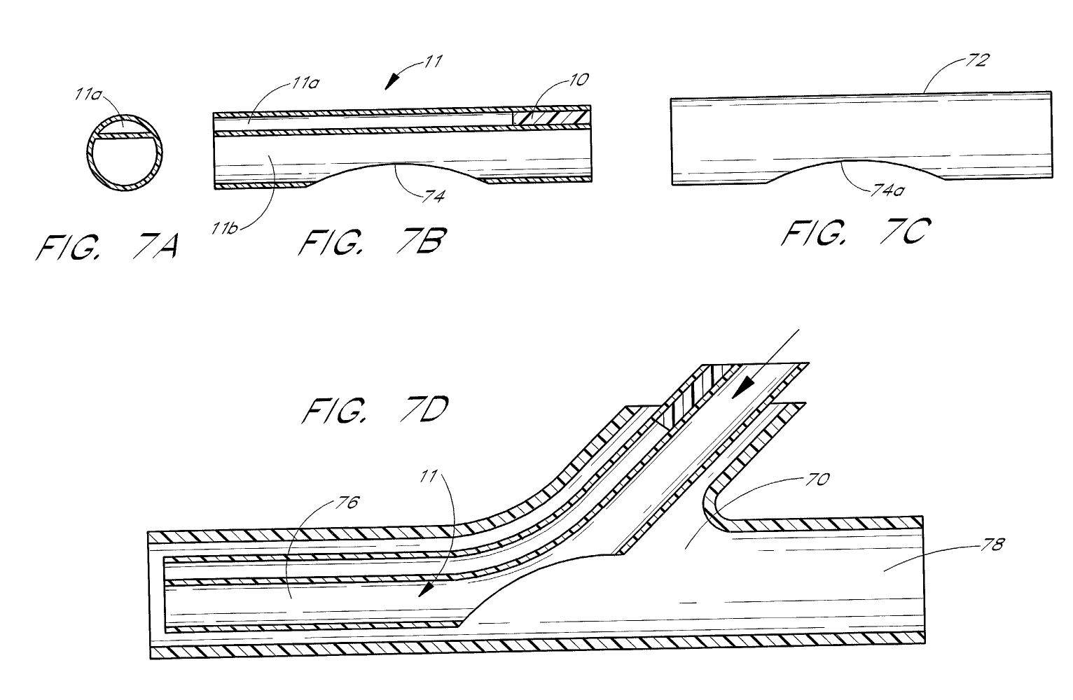

FIGS. 7A, 7B, 7C and 7D show a perfusion catheter 11 with two lumens and an adaptor used for the catheter in accordance with one embodiment of the present invention. The catheter 11 has two lumens 11a and 11b. One of the lumens 11a is in fluid communication with a distal balloon and is plugged at the proximal end of the catheter 11 with any suitable plug as described previously. The other lumen 11b is for perfusion (blood/drug) which has a relative large opening 74 on side wall of catheter 11. A flexible tube 72, such as a Nitinol tube, has an opening 74a on its side wall similar to opening 74 in size and shape. Tube 72 is slid over catheter 11 forming an assembly 76 with opening 74 and 74a aligned with each other. The assembly 76 is inserted into a Y-shaped adaptor 70. The passage 78 formed between opening 74, 74a and the Y-shaped adaptor is used to introduce a perfusion fluid.

In order to be able to deliver an accurate amount of an inflation fluid to the balloon, a variety of low volume syringes to be used with the plug system although, relatively large volume syringes can also be used in the plug system. Other syringes compatible with the principles of the present invention are described in assignee's co-pending U.S. application Ser. No. ______ (Attorney Docket PERCUS.023CP1) filed on ______, which is entitled SYRINGE AND METHOD FOR INFLATING LOW VOLUME CATHETER BALLOON, the entirety of which is hereby incorporated by reference.

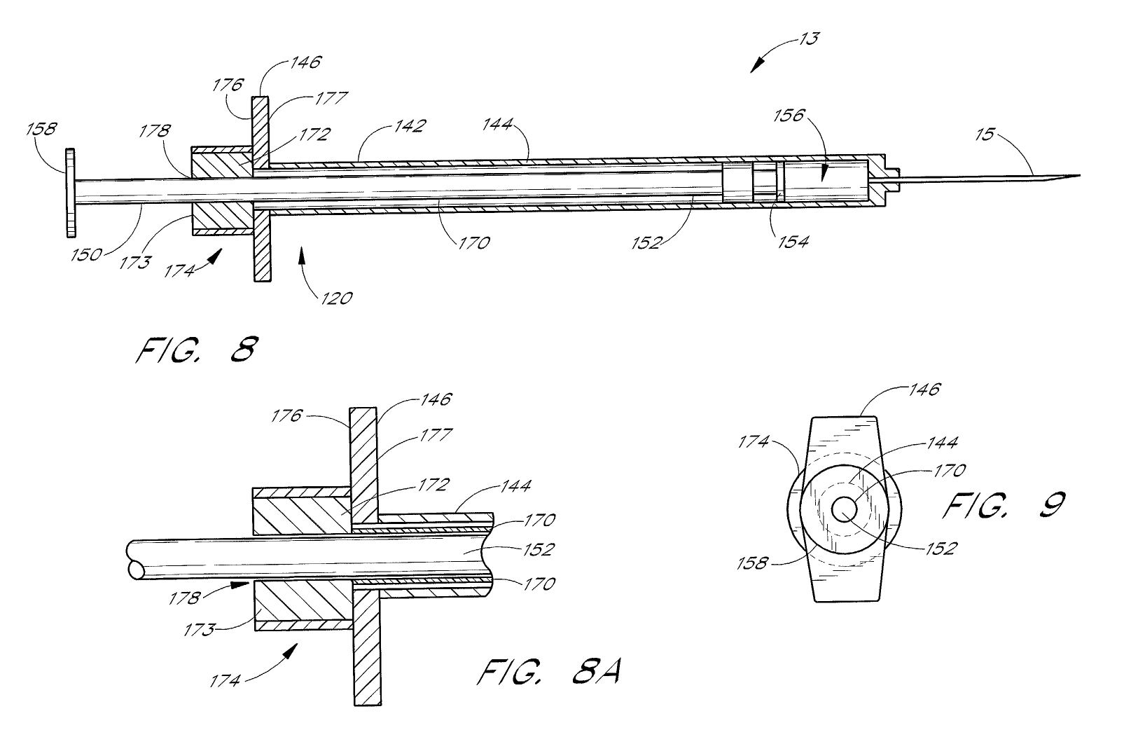

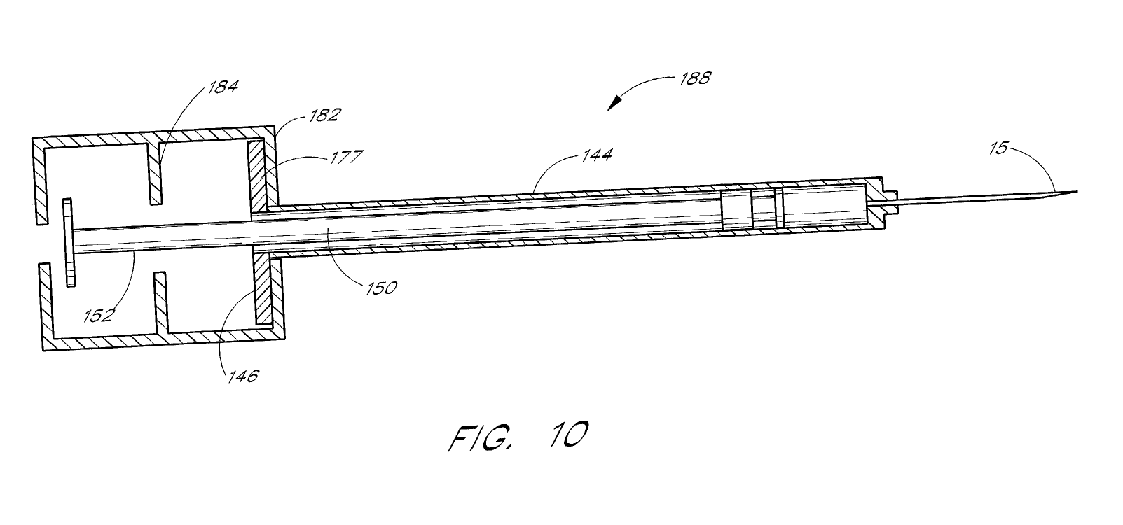

A low volume syringe 13 is shown schematically in FIG. 8. The type or size illustrated is a 0.5 cc tuberculin syringe, although other size syringes may be used. The resultant displacement required for delivery of about 0.1 cc fluid is about 10 mm for a 0.25 cc syringe. Indicia 142 may be provided along the length of the exterior surface of a cylinder 144 for visual aid of the clinician during use. However, as described below in more detail, a stop mechanism is advantageously provided on the syringe 13 in order to accurately limit the inflation fluid intake and expulsion, thereby providing a means for the clinician to safely and accurately perform the desired procedure.

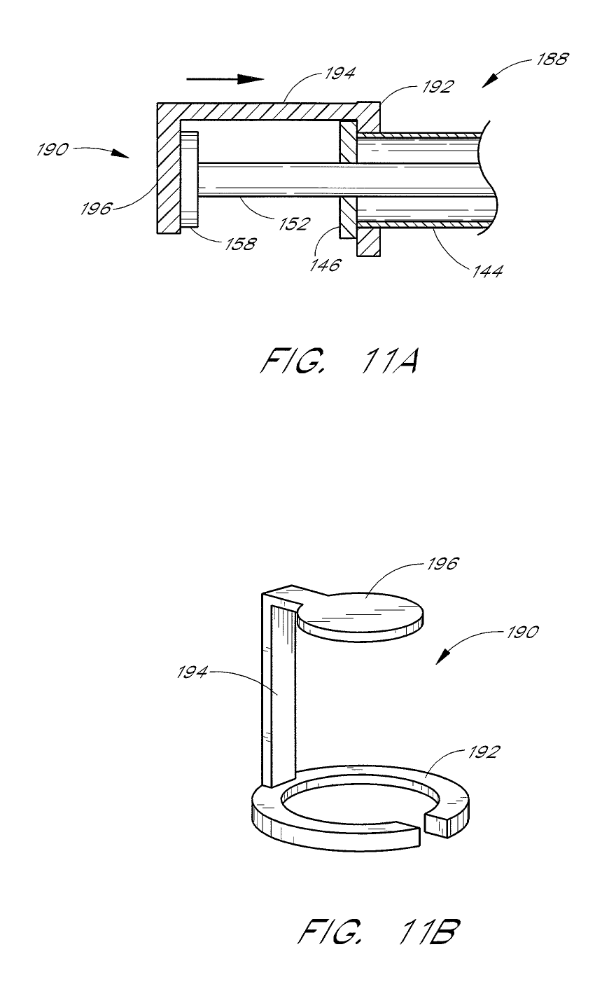

Referring to FIGS. 8 and 9, the elongate body of the syringe comprises a cylinder 144 having a stop or flange 146 extending radially outward at a proximal end and being attached at a distal end to needle 15. Preferably, injection guide 14 (not shown) can be provided surrounding the needle 15 in a manner as described previously. The distal end of the cylinder 144 has a nose portion 148 with a reduced diameter for connection with the needle 15. A plunger 150 has a shaft 152 of appropriate length and a resilient piston 154 attached at its distal end. The shaft 152 is inserted in a central lumen 156 of the cylinder and the piston 154 provides sealing engagement with the inner surface of the cylinder 141. The plunger 150 has a disk 158 at the proximal end of the shaft 152 for operation of the plunger 150. A preferred source for unmodified, conventional syringes is Becton Dickinson & Co. of Franklin Lakes, N.J.

In the embodiment of FIGS. 8 and 9, the limiting of the fluid intake and expulsion is accomplished by a tube 170 on the plunger 150 which are contained within the lumen 156 of the cylinder 144. The tube 170 has a length shorter than the lengths of the cylinder 144 and the shaft 152 and this length determines the volumetric intake and expulsion of the fluid from the syringe. The inner diameter of the tube 70 is preferably sized to be approximately the same as the outer diameter of the plunger shaft 152. The tube 170 is circumferentially attached to the plunger shaft 152, as shown in FIG. 9. Preferably, an adhesive, such as LOCTITE (TM) 4011 is used to secure the tube 170 to the shaft 152; although, attachment of the tube 170 to the plunger shaft 152 is not required to achieve the benefits of the present invention.

In use, for intake of the inflation fluid, a proximal end of the tube 170 contacts a distal face 172 of an insert within the barrel 174, as shown in more detail in FIG. 8A, thus providing a stop mechanism for limiting the intake of inflation fluid (and thereby limiting the amount of fluid which is available for inflation). The barrel 174 is attached to a proximal face 176 of the flange 146 and thereby limits the plunger's outward or proximal travel with respect to the cylinder 144. The barrel 174 has a central channel 178 sized to allow the plunger shaft 152 to slide through, but not the plunger shaft 152 having the tube 170 surrounding it. Preferably, the distal end of the barrel 174 is secured to the face 176 of the flange 146 using adhesive. Alternatively, the tube 170 and barrel 174 may be integrally formed with the plunger 150 and flange 146, respectively, of the syringe 13.

It will be noted from FIG. 8 that the proximal face 173 of the barrel 174 can be positioned a predetermined distance from the disk 158. In other words, the length of the barrel 174 can be varied so that the proximal surface 173 thereof may also serve as a stop mechanism, alone or in combination with action of the tube 170.

For the 0.5 cc syringe shown, preferred dimensions of the tube 170 include an OD of about 0.135″ and an ID of about 0.127″, with a tube length of about 115 mm. The preferred tube material is nylon 12. The corresponding preferred dimensions of the barrel 174 are an OD of about 0.288″ and an ID of about 0.128″, with a barrel length of about 0.260″. The preferred barrel material is a polycarbonate.

An alternative embodiment of the low volume syringe is shown in FIG. 10, wherein a conventional syringe 188 is retro-fit with a stop member 180 that is attached over the proximal end of the syringe. The member 180 includes an attachment portion 182 and two limiting portions 184, 186. The attachment portion 182 is at the distal end of the member 180 and may be secured to the distal face 177 of the cylinder flange 146 using adhesive, or it may be attached to the outer surface of the cylinder 144. The limiting portion 186 at the proximal end of the member extends proximally past the plunger disk 158, or in a direction away from the plunger shaft 152. At least this portion 186 should be limited to two segments of an annular disk of no more than about 45 degrees each, for example, to allow a proximal opening sufficient for a thumb or the like to access the plunger disk 158. The other limiting portion 184, intermediate the other portions 182, 186, is positioned between the cylinder flange 146 and the plunger disk 158. Thus, the limiting portions 184, 186 form the intake and delivery stops for the modified syringe 188; although, in alternative embodiments, the portion 184 may be omitted without loss of benefit of the present invention.

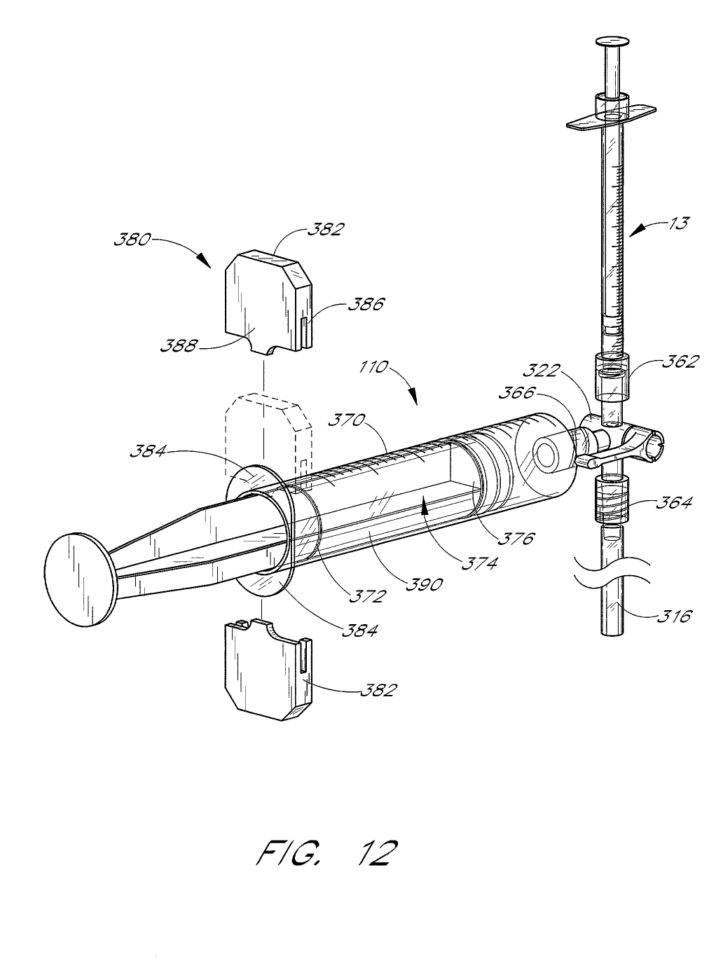

Another embodiment of a retro-fit stop member 190 for a conventional syringe is shown in FIGS. 11A and 11B. A preferably C-shaped attachment disk 192 has an arm 194 extending transverse to its diameter and parallel to the length of the syringe cylinder 144. A smaller disk 196 is provided at right angles to the arm 1911 and is substantially parallel to and aligned over the C-shaped disk 192. The opening of the C-shaped disk 192 allows the member 190 to be snapped into place over the distal end of the syringe cylinder 144 and is preferably left free to slide thereover. The smaller disk 196 is preferably glued to the outer face of the plunger disk 158. Alternatively, the C-shaped disk 192 could be glued into place on the cylinder 144, and the smaller disk 196 could be left free to engage and disengage the plunger disk 158 during use. Thus, the intake or travel of the plunger 150 in this modified syringe is limited by the length of the arm 194 of the stop member 190.

Another arm (not shown) comprising a member extending parallel to the C-shaped disk 192 and smaller disk 196 and provided along the longitudinal arm 194 between the two disks 192, 196 may be provided to limit the amount delivered by the syringe, in a manner similar to FIG. 10. Although, if the predetermined amount of fluid to be drawn into the inflation syringe is to be the same as the predetermined amount of fluid to be delivered or injected by the syringe, then the construction shown in FIGS. 11A and 11B accomplishes this goal.

The stop member 120, 180, 190 is preferably made of a resilient material and manufactured in various lengths. The member may be integrally formed, as a retro-fit mechanism or directly on the syringe, or assembled from smaller components. As a retro-fit, the stop member may have alternative configurations that can be hinged, snapped or clipped into place, for example, over the proximal end of the syringe. The retro-fitting is also suitable for providing advantages of the low volume syringe of the present invention to syringes comprising larger diameter plunger barrels that preclude the use of a tube 170 in the cylinder lumen 156.

FIG. 12 illustrates a dual syringe system that can withstand the initial build up pressure. Although this figure illustrates a dual syringe system, this by no means limits the invention to this configuration. For example, the high pressure capability syringe system can be incorporated in conjunction with a single syringe system.

During the inflation of an occlusion or surgical balloon, the pressure in the system adjacent to the syringe 13 can reach pressures up to 200 psi. This pressure build up is caused by the sudden volume change caused by the influx of fluid into the balloon and lines leading to the balloon. The build up pressure will peak initially and then dissipate as the pressure disperses through the lines toward the balloon. Eventually, the build up pressure will dissipate when the balloon inflates. Typically, the build up pressure peaks and then stabilizes within 5 seconds. Therefore, the syringe system must be able to withstand the peak pressure for at least 5 seconds.

In the dual syringe system of FIG. 12, a large volume or reservoir syringe 110 is attached and in fluid communication with a three-way high-pressure stop cock valve 322. The syringe 110 provides the desirable power and volume for quickly priming the balloon and any involved lines, lumens, valves, or guidewire, as well as for quickly deflating the balloon. A preferred provider of the high pressure valve is Merit Medical Systems part number M3SNP. Preferably, the valve is rated to handle pressures of 250 psi. Even more preferably, the valve 322 should be rated to withstand a pressure of 500 psi. Luer type couplings extend from all sides of the valve 322. Preferably the coupling 262 includes an extended engagement area, namely a greater number of threads, to ensure the positive sealing between the low volume syringe 13 and the valve 322. Likewise, the syringe 13 includes a mating, engagement area to complete the connection between the syringe 13 and the fitting 362. Although not illustrated, an O-ring could be incorporated between the mating surfaces of the syringe 13 and the fitting 362 to insure a leak-free seal.

A similar high pressure fitting 364 is located on the opposing side of the high pressure stop cock valve 322. As before, this fitting 364 has an extended engagement area, i.e. more threads, to lessen the load caused by the high build up pressure. Attached to the high pressure fitting 364 is a high pressure line 316 leading either to a needle, an inflation device, or directly to the balloon (not shown in this figure). Alternatively, a needle can be directly attached to the high pressure fitting 364. Preferably, the high pressure line 316 is rated to withstand a pressure of 250 psi. Even more preferably, the line 316 is rated to withstand a pressure of 500 psi. An example of the line is part number 70078 manufactured by Mallinkrodt.

Still referring to FIG. 12, a similar high pressure fitting 366 is located on the remaining side of the high pressure stop cock valve 322. This fitting 366 preferably is a luer type connector with an extended engagement area, i.e. more threads, to lessen the load caused by the high build up pressure. Attached to the high pressure fitting 366 is a large volume reservoir syringe 110 which has a mating connector at a distal end which mates with the high pressure fitting 366 to insure a fluid tight seal.

In addition to providing a highly responsive inflation system for an occlusion balloon the dual syringe system also has a variety of other uses. For instance, the system could be used to deliver precise amounts of therapeutic drugs or medicine to the patient. The system may also be used to irrigation or aspiration. Additionally, the system can be used to infuse whole blood as is described below.

Typically, whole blood is infused into patients with roller type pumps. One problem associated with this type of pump is that roller mechanisms apply a shear stress that often damages the blood cells with the crushing force of the rollers. The dual syringe system could overcome the problem of damaging the blood by providing a hydrostatic pressure that would provide pressure for the transfusion without causing the damaging forces on the cells. The blood cells, because of their circular shape, can withstand great hydrostatic pressure and therefore would not be damaged. Preferably, the large volume syringe will be used to infuse blood.

In use, a guidewire or a balloon catheter with the plugging and sealing mechanism of the present invention is first inserted into a vascular system and advanced to the treatment site. In order to inflate the balloon on the distal end of the catheter, a syringe, preferably a syringe of the present invention, is used to deliver an inflation fluid. The needle of the syringe is inserted through the plugging or sealing material into the lumen of the catheter and inject the inflation fluid. At this stage, the syringe can be provided with an injection guide or can be used with an adaptor as previously described. After the balloon is inflated, the syringe is removed from the catheter and the catheter is resealed by the plugging material due to its elasticity. At this step, another catheter can be slid over the original catheter without removing the plugging mechanism while keeping the pressure inside the balloon catheter.

The embodiments of the apparatus and method as described above are provided merely to illustrate the present invention. Changes and modifications may be made from the embodiments presented herein by those skilled in the art without departure from the spirit and scope of the invention, as defined by the appended claims.

What is claimed is:

1. A plug system for a fluid delivery catheter, the catheter having a distal portion and a proximal portion and at least one lumen in fluid communication therebetween, the catheter having an outer diameter in the range of about 0.01″ to 0.041″, and an inner diameter in the range of about 0.007″ to 0.035″, the plug system comprising a pierceable, self-sealable plug for engaging and sealing said proximal portion of the catheter in such a way that the maximum outer diameter of the plug does not exceed 140% of the outer diameter of said catheter.

2. The plug system of claim 1, wherein the plug is molded into the open end.

3. The plug system of claim 1, wherein the plug is pre-made and glued onto the open end.

4. The plug system of claim 1, wherein the catheter has an outer diameter in the range of about 0.01″ to 0.021″, and an inner diameter in the range of about 0.007″ to 0.017″.

5. The plug system of claim 1, further comprising an injection guide having an aperture for receiving the open end of the catheter, and a syringe having a needle for penetrating the plug and delivering an inflation fluid to the lumen, wherein the aperture of the injection guide is positioned surrounding the needle so that the injection guide will guide the needle to the plug.

6. A low profile fluid delivery and sealing system for a catheter with a lumen, comprising a pierceable and self-resealable member sealably coupled to the lumen at a proximal end of the catheter forming an assembly, wherein the transverse cross sectional area of the assembly as viewed along a longitudinal axis of the catheter does not exceed 150% of the original traverse cross sectional area of the catheter, and that the lumen is accessible through the member.

7. The system of claim 6, wherein the catheter has an outer diameter in the range of about 0.01″ to 0.041″, and an inner diameter in the range of about 0.007″ to 0.035″.

8. The system of claim 6, wherein the catheter has an open end at its very proximal end, the member has a cylindrical body and a flange extending from the periphery of the cylindrical body along a longitudinal axis of the cylindrical body, wherein the flange defines a space for receiving the open end of the catheter, and the thickness of the cylindrical solid body as measured along the longitudinal axis is large enough to provide a good sealing after a needle is inserted through and removed from the member.

9. The system of claim 8, wherein the thickness of the flange does not exceed 0.005″.

10. The system of claim 6, wherein the catheter has an opening on its side wall near the proximal end, and the opening is covered and sealed by the member, wherein the member comprises a sealing membrane with a thickness less than 0.005″.

11. The system of claim 10, further comprising an adaptor for guiding a needle to the opening, wherein the adaptor has a locking mechanism for holding and positioning the adaptor onto the catheter, and a guiding channel aligned with the opening for guiding the needle to the opening.

12. The system of claim 6, wherein the member comprises a tube connected to the catheter at its two ends and forms a portion of the catheter.

13. The system of claim 6, further comprising a syringe for injecting a predetermined amount of fluid into the catheter, wherein the syringe comprises:
an elongated cylinder forming a lumen and having a proximal end and a distal end, a finger stop formed about the proximal end and extending outwardly from the lumen, the distal end adapted to attach to a proximal portion of the catheter;
a plunger for use in the cylinder and having a shaft with a disk provided at a proximal end and a piston provided at a distal end of the shaft, the disk having a diameter larger than a diameter of the shaft, the piston having a diameter substantially the same as a diameter of the lumen of the cylinder;
a barrel provided proximal the finger stop at the proximal end of the cylinder, the barrel having a length and a longitudinal channel formed therethrough, the channel having a diameter sized to receive the shaft of the plunger; and
a tube provided at a proximal end of the lumen, the tube having an outer diameter less than the diameter of the lumen and an inner diameter forming a longitudinal channel, the inner diameter sized to receive the shaft of the plunger;
wherein the tube has a length for limiting the proximal travel of the plunger and thereby limiting a maximum volume of fluid that can be contained in the syringe, the barrel having a length for limiting the distal travel of the plunger and thereby limiting a maximum volume of fluid that can be expelled from the syringe.

14. The system of claim 13, further comprising an injection guide having an aperture for receiving the open end of the catheter, wherein the aperture of the injection guide is positioned surrounding the needle of the syringe so that the injection guide will guide the needle to the plug.

15. The system of claim 6, further comprising a dual syringe system for delivering fluid into the catheter or removing fluid therefrom, wherein the dual syringe system comprises:
a low volume syringe;
a large volume syringe;
a high pressure valve assembly;
a high pressure fluid conduit;
wherein said high pressure valve assembly is in fluid communication with said low volume syringe, said large volume syringe and said high pressure fluid conduct, and selectively allows fluid communication between;
a) said large volume syringe and said low volume syringe, or;
b) said low volume syringe and said high pressure fluid conduit, or;
c) said large volume syringe and said high pressure fluid conduit.

16. A method for inflating/deflating a balloon on a guide catheter and exchanging a treatment catheter, the guide catheter having an opening connected to a lumen in fluid communication with the balloon, comprising the steps of:
a) providing an elastomeric member to seal the opening of the guide catheter;
b) providing a syringe with a needle;
c) inserting the needle into the lumen through the elastomeric member and the opening of the guide catheter so as to establish fluid communication between the lumen and the syringe;
d) injecting a fluid into the lumen to inflate the balloon;
e) removing the needle to allow the elastomeric member to self-seal so that the balloon is kept inflated;
f) advancing a treatment catheter to or removing a treatment catheter from a treatment site over the guide catheter.

17. The method of claim 16, wherein the treatment catheter has an inner diameter in the range of about 0.007-0.035″.
